(19) United States
(12) Patent Application Publication
(10) Pub. No.: US 2022/0395436 A1
(43) Pub. Date: Dec. 15, 2022

(54) GEL COMPOSITION WITH HIGH OIL CONTENT, THE PREPARATION METHOD AND THE USE OF THE SAME

(71) Applicant: BASF SE, Ludwigshafen (DE)

(72) Inventors: Zhi Ting Jiang, Shanghai (CN); Long Zhao, Shanghai (CN); Yu Ting Xu, Shanghai (CN); Yi Yao Wang, Shanghai (CN)

(21) Appl. No.: 17/636,056

(22) PCT Filed: Sep. 17, 2020
(86) PCT No.: PCT/CN2020/115784

§ 371 Date: Feb. 17, 2022

(30) Foreign Application Priority Data
Sep. 18, 2019 (CN) PCT/CN2019/106431

(51) Int. Cl. A61K 8/04; A61K 8/34; A61K 8/60; A61K 8/37; A61Q 1/14

(52) U.S. Cl. CPC A61K 8/042; A61K 8/34; A61K 8/602; A61K 8/37; A61K 8/375; A61Q 1/14; A61K 2800/48

(57) ABSTRACT

It relates to a cosmetic composition in the form of a transparent or semi-transparent gel, comprising (a) an oil phase; (b) a surfactant portion comprising a first non-ionic surfactant and a second non-ionic surfactant; (c) an aqueous phase; and (d) at least one C₂-C₆ polyol; wherein the first nonionic surfactant comprises at least one alkyl polyglucoside and the second nonionic surfactant comprises at least one having the following general formula (I) or (II): R(X)ₙOR′ (I) wherein, R represents a linear or branched C₈-C₃₀ alkyl group or C₈-C₃₀ alkenyl group; X represents —OCH₂CH₂— or —OCH₂—CHCH₃—; n represents an integer from 2 to 100; and R′ represents H, or a linear or branched C₈-C₃₀ alkyl group or C₈-C₃₀ alkenyl group; (II) wherein, the sum of x+y+z represents an integer from 2 to 100; and R₁, R₂, and R₃, are the same or different and are each independently represents OH, or a linear or branched C₈-C₃₀ fatty acid ester group, with the proviso that at least one of R₁, R₂ and R₃ is not OH. It also relates to a method for preparing the cosmetic composition and the use thereof. The cosmetic composition has high oil content, superior stability and desirable cleansing efficacy.

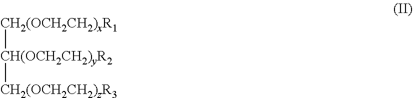

### TECHNICAL FIELD

The present invention relates to cosmetic compositions in the form of a transparent or semi-transparent gel, the preparation methods and the uses thereof.

### BACKGROUND OF THE INVENTION

In the cosmetics and personal care product market, the currently existing products, such as makeup removers, are mainly classified into four types of forms, oil form, water form, cream (emulsion) form, and gel form. Makeup removal oils typically contain a large amount of oil, and can efficiently dissolve and remove the various fatty substances present on the skin or derived from the make-up such as lipsticks, mascaras and foundations. However, they require such packaging as to accommodate their high oil level, and are usually used before other cleansing products, which remove the greasy residue on the skin. Make-up remover cleansing water, like micellar water, contains water as the main ingredients together with a certain amount of surfactants. It is known that these kinds of cleansing water cannot efficiently remove cosmetics of oil base formulation. Compared with the cleansing water, makeup removal cream (emulsions) includes a small amount of oil, and thus can remove cosmetics to some extent but still cannot achieve the desired makeup removing effect. Make-up removal gels include water gel and oil gel formulation, the former one being normally thickened by polymeric thickener. Oil gel can be further classified into two subtypes: one subtype which is thickened for the oil base by polymeric thickeners, like ethylene/propylene/styrene copolymer, dibutyl ethylhexanoyl glutamide, dextrin palmitate, etc., and the other subtype which develops viscosity by its own structure without thickener.

Gel products with high oil content have been widely used in personal care and cosmetics industry, because they can overcome some drawbacks or limitations of the liquid oil formulations, such as the inconvenience owing to the high flowability and the high demands on packaging owing to the compatibility of oil. For the purposes of easy use and desirable stability, the currently available gel products with high oil content are usually thickened by oil phase thickeners or through the method of D-phase emulsification. Nevertheless, gel products obtained by using oil phase thickeners, also have such problems as poor spreadability and unpleasant sensory feeling including an oily and sticky feeling of heaviness or retention of an oil film on the skin; and another kind of gel cream products obtained by using D-phase emulsification often suffers instability and usually appears as a milk-white cream, which is not pleasing and commercially attractive for consumers.

Accordingly, it remains a challenge to obtain stable, transparent or semi-transparent, and easy to spread gel products with high oil content.

Currently, a gel product can be easily prepared but it is difficult to prepare a stable one and maintain the stability during storage. The existing solution for making a gel product stable is adding gelling agent or thickening polymer (also known as polymeric thickener). For example, U.S. Pat. No. 6,524,594B1 (Delores M. Santora et al.) discloses a gelled oil composition comprising an emulsifier, a gelling agent, an oil and a surfactant. It discloses that the gelling agent accounts for from about 3% to about 10% and preferably from about 3.5% to about 5.5%, based on the total weight of the composition. The gelling agent disclosed in U.S. Pat. No. 6,524,594B1 is selected from dextrin myristate, dextrin palmitate, a blend of at least two different polymer members selected from the group consisting of diblock copolymers, triblock copolymers, radical block copolymers and multiblock copolymers, or a mixture thereof. Similarly, WO2016034521A1 (Calvert et al.) also discloses a transparent gel base comprising a polymeric thickener that is responsible for the gelled structure of the composition. The polymeric thickeners disclosed in WO2016034521A1 include hydrophobically modified ethoxylated urethane (HEUR), carbomers, polyvinylpyrrolidone (PVP), cross linked vinyl pyrrolidone copolymers and copolymers of vinyl methyl ether and maleic anhydride. The presence of the gelling agent or the polymeric thickener in the above-disclosed gel products makes them less spreadable and not pleasant after being applied to the skin.

### SUMMARY OF THE INVENTION

In the process of developing stable and attractive personal care products, which overcome at least one of the above-mentioned problems with the current personal care products, the inventors have surprisingly found that the combination of two kinds of non-ionic surfactants—alkyl polyglucosides and polyoxyalkylene alkyl ethers or polyoxyethylene fatty acid glyceryl esters—will give an oil-in-water emulsion of gel appearance, an emulsion which is transparent or semi-transparent, attractive to consumers, and easy to package and transport. Moreover, the gel composition of the invention can be free or substantially free of polymeric thickener, gelling agent or thickening polymer. Despite the absence or substantial absence of polymeric thickener, gelling agent or thickening polymer, the gel products of the invention with high oil content still have superior stability, satisfactory spreadability when being applied and desirable cleansing efficacy.

In one aspect, the invention provides a cosmetic composition, comprising
(a) an oil phase;
(b) a surfactant portion comprising a first non-ionic surfactant and a second non-ionic surfactant, wherein the first nonionic surfactant comprises at least one alkyl polyglucoside and the second nonionic surfactant is at least one selected from surfactant having the following general formula (I) or (II):

R(X)ₙOR′ (I)
wherein, R represents a linear or branched C₈-C₃₀ alkyl group or C₈-C₃₀ alkenyl group;
   X represents —OCH₂CH₂— or —OCH₂—CHCH₃—;
   n represents an integer from 2 to 100; and
   R′ represents H, or a linear or branched C₈-C₃₀ alkyl group or C₈-C₃₀ alkenyl group;

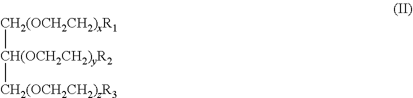

wherein, the sum of x+y+z represents an integer from 2 to 100;
and R₁, R₂, and R₃, are the same or different and each independently represents OH, and/or a linear or branched C₈-C₃₀ fatty acid ester group, with the proviso that at least one of R₁, R₂ and R₃ is not OH;
(c) an aqueous phase; and
(d) at least one C₂—C polyol;
wherein said composition is in the form of a transparent or semi-transparent gel.

In another aspect, the invention provides a method for preparing the above-mentioned cosmetic composition, comprising the following steps:
(a) adding the surfactant portion, the C₂-C₆ polyol to the aqueous phase, and mixing—optionally and preferably with heating, stirring or simultaneous heating and stirring—them uniformly;
(b) heating the oil phase;
(c) adding the oil phase obtained in step (b) into the aqueous phase obtained in step (a) under stirring; and
(d) cooling the mixture obtained in step (c) under stirring to give a gel.

In a third aspect, the invention provides use of the above-mentioned cosmetic composition for the preparation of personal care products.

In a fourth aspect, the invention provides a method for personal care or cleansing, comprising the step of applying the cosmetic composition of the invention to a desired location on the skin or hair in the presence of water, and then rinsing off the composition from the skin or hair with water.

The cosmetic composition of the invention is in the form of a transparent or semi-transparent gel, which is attractive to consumers. Moreover, the cosmetic composition in the gel form of the invention has superior stability and spreadability, although it has high level of oil.

As the cosmetic composition of the invention is in the form of a transparent or semi-transparent gel, it may also be referred to as “gel composition(s) of the invention”, “gel product(s) of the invention” or “gel(s) of the invention”. These terms will be used interchangeably throughout the present description and the appended claims.

The aspects of the present invention will become apparent from the detailed description provided hereinafter. It should be understood that the detailed description and the specific examples, while indicating the preferred embodiment of the invention, are intended for purposes of illustration only and are not intended to limit the scope of the invention.

### DETAILED DESCRIPTION OF THE INVENTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. Unless otherwise specified, all percentages and amounts expressed herein and elsewhere in the description should be understood to refer to percentages by weight. For example, the symbol “wt %” means percentages by weight. The amounts given are based on the active weight of the material.

In one aspect, the invention provides a cosmetic composition, comprising
(a) an oil phase;
(b) a surfactant portion comprising a first non-ionic surfactant and a second non-ionic surfactant, wherein the first nonionic surfactant comprises at least one alkyl polyglucoside and the second nonionic surfactant is at least one selected from surfactant having the following general formula (I) or (II):

R(X)ₙOR′ (I)
wherein, R represents a linear or branched C₈-C₃₀ alkyl group or C₈-C₃₀ alkenyl group;
   X represents —OCH₂CH₂— or —OCH₂—CHCH₃—;
   n represents an integer from 2 to 100; and
   R′ represents H, or a linear or branched C₈-C₃₀ alkyl group or C₈-C₃₀ alkenyl group;

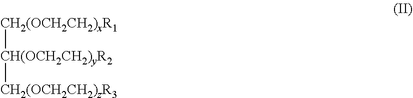

wherein, the sum of x+y+z represents an integer from 2 to 100;
and R₁, R₂, and R₃, are the same or different and each independently represents OH, and/or a linear or branched C₈-C₃₀ fatty acid ester group, with the proviso that at least one of R₁, R₂ and R₃ is not OH;
(c) an aqueous phase; and
(d) at least one C₂—C polyol;
wherein said composition is in the form of a transparent or semi-transparent gel.

In an embodiment, based on the total weight of the cosmetic composition,
(1) the oil phase may be present in an amount of from 50 wt % to 90 wt %, such as from 55 wt % to 89 wt %, from 60 wt % to 88 wt %, from 65 wt % to 87 wt %, from 70 wt % to 86 wt %, from 75 wt % to 85 wt %;
(2) the surfactant portion comprising the first non-ionic surfactant and the second non-ionic surfactant may be present in an amount of from 1 wt % to 20 wt %, such as from 1.5 wt % to 18 wt %, from 2 wt % to 16 wt %, from 2.5 wt % to 14 wt %, from 3 wt % to 12 wt %, from 3.5 wt % to 10 wt %, from 4 wt % to 9 wt %, from 4.5 wt % to 8.5 wt %, from 5 wt % to 8 wt %, from 5.5 wt % to 7.5 wt %, from 6 wt % to 7 wt %;
(3) the aqueous phase may be present in an amount of from 1 wt % to 15 wt %, such as from 2 wt % to 12 wt %, from 3 wt % to 11 wt %, from 4 wt % to 10 wt %, from 5 wt % to 9 wt %, from 6 wt % to 8 wt %; and
(4) the C₂-C₆ polyol may be present in an amount of from 1 wt % to 15 wt %, such as from 2 wt % to 12 wt %, from 3 wt % to 10 wt %, from 4 wt % to 8 wt %, and from 5 wt % to 7 wt %.

In an embodiment, in the surfactant portion, the first non-ionic surfactant and the second non-ionic surfactant may be present at the weight ratio in the range of 9:1 to 1:9, such as in the range of 8:2 to 2:8, in the range of 7:3 to 3:7, or in the range of 6:4 to 4:6, and particularly at the weight ratio of 9:1, 8:2, 7:3, 6:4, 5:5, 4:6, 3:7, 2:8 or 1:9. In addition, the first non-ionic surfactant and the second non-ionic surfactant may be present at weight ratio in any sub-range within the above ranges or in any sub-range having the above specific ratio values as the terminus, such as in the range of 9:1 to 2:8, in the range of 9:1 to 3:7, in the range of 9:1 to 4:6, in the range of 9:1 to 5:5, in the range of 9:1 to 6:4, in the range of 9:1 to 7:3, in the range of 8:2 to 1:9, in the range of 8:2 to 3:7, in the range of 8:2 to 4:6, in the range of 8:2 to 5:5, in the range of 8:2 to 6:4, in the range of 8:2 to 7:3, in the range of 7:3 to 1:9, in the range of 7:3 to 2:8, in the range of 7:3 to 4:6, in the range of 7:3 to 5:5, in the range of 7:3 to 6:4, in the range of 6:4 to 1:9, in the range of 6:4 to 2:8, in the range of 6:4 to 3:7, in the range of 6:4 to 5:5, in the range of 5:5 to 1:9, in the range of 5:5 to 2:8, in the range of 5:5 to 3:7, in the range of 5:5 to 4:6, in the range of 4:6 to 1:9, in the range of 4:6 to 2:8, in the range of 4:6 to 3:7, in the range of 3:7 to 1:9, in the range of 3:7 to 2:8, or in the range of 2:8 to 1:9.

In an embodiment, based on the total weight of the cosmetic composition, the first non-ionic surfactant may be present in an amount of from 0.5 wt % to 19.5 wt %, such as from 0.6 wt % to 18 wt %, from 0.7 wt % to 16 wt %, from 0.8 wt % to 14 wt %, from 0.9 wt % to 12 wt %, from 1 wt % to 10 wt %, from 1.1 wt % to 8 wt %, from 1.2 wt % to 7 wt %, from 1.3 wt % to 6 wt %, from 1.4 wt % to 5 wt %, from 1.5 wt % to 4.8 wt %, from 1.6 wt % to 4.5 wt %, from 1.8 wt % to 4.2 wt %, from 2 wt % to 4 wt %, from 2.4 wt % to 3.6 wt %, from 2.5 wt % to 3 wt %, 2.5 wt %, 2.6 wt %, 2.7 wt %, 2.8 wt %, 2.9 wt %, 3.0 wt %, 3.1 wt %, 3.2 wt %, 3.3 wt %, 3.4 wt % and 3.5 wt %; and the second non-ionic surfactant is present in an amount of from 0.5 wt % to 19.5 wt %, from 0.6 wt % to 18 wt %, from 0.7 wt % to 16 wt %, from 0.8 wt % to 14 wt %, from 0.9 wt % to 12 wt %, from 1 wt % to 10 wt %, from 1.1 wt % to 8 wt %, from 1.2 wt % to 7 wt %, from 1.3 wt % to 6 wt %, from 1.4 wt % to 5 wt %, from 1.5 wt % to 4.8 wt %, such as from 1.6 wt % to 4.5 wt %, from 1.8 wt % to 4.2 wt %, from 2 wt % to 4 wt %, from 2.4 wt % to 3 wt %%, 2.5 wt %, 2.6 wt %, 2.7 wt %, 2.8 wt %, 2.9 wt %, 3.0 wt %, 3.1 wt %, 3.2 wt %, 3.3 wt %, 3.4 wt % and 3.5 wt %.

In an embodiment, the alkyl polyglucoside may be one or more selected from the group consisting of C₈-C₁₀ alkyl polyglucosides, C₈-C₁₆ alkyl polyglucosides, C₁₂-C₁₆ alkyl polyglucosides, C₉-C₁₁ alkyl polyglucosides, C₁₂-C₂₀ alkyl polyglucosides, and a combination thereof. More particularly, the alkyl polyglucoside may be one or more selected from the group consisting of decyl glucoside, arachidyl glucoside, caprylyl/capryl glucoside, cetearyl glucoside, coco-glucoside, lauryl glucoside, and a combination thereof.

In an embodiment, the alkyl polyglucoside may be commercially available, for example as APG®, GLUCOPON®, or PLANTAREN® surfactants from Cognis Corporation (Ambler, Pa., 19002), or PLANTACARE® surfactants from BASF. Examples of such surfactants may include but are not limited to:
1. GLUCOPON® 225DK Surfactant—an alkyl polyglucoside in which the alkyl group contains 8 to 10 carbon atoms and having an average degree of polymerization of 1.7;
2. GLUCOPON® 425N Surfactant—an alkyl polyglucoside in which the alkyl group contains 8 to 16 carbon atoms, having an average of 10.3 carbon atoms, and having an average degree of polymerization of 1.5;
3. GLUCOPON® 625UP Surfactant—an alkyl polyglucoside in which the alkyl group contains 12 to 16 carbon atoms and having an average degree of polymerization of 1.6;
4. APG® 325N Surfactant—an alkyl polyglucoside in which the alkyl group contains 9 to 11 carbon atoms and having an average degree to polymerization of 1.5;
5. GLUCOPON® 600UP Surfactant—an alkyl polyglucoside in which the alkyl group contains 12 to 16 carbon atoms and having an average degree of polymerization of 1.4;
6. PLANTAREN©2000 Surfactant—a C₈-C₁₆ alkyl polyglucoside in which the alkyl group contains 8 to 16 carbon atoms and having an average degree to polymerization of 1.5;
7. PLANTAREN®1300 Surfactant—a C₁₂-C₁₆ alkyl polyglucoside in which the alkyl group contains 12 to 16 carbon atoms and having an average degree to polymerization of 1.6;
8. GLUCOPON® 220N Surfactant—an alkyl polyglucoside in which the alkyl group contains 8 to 10 carbon atoms and having an average degree of polymerization of 1.5;
9. PLANTACARE® 818 UP Surfactant—a C₈-C₁₆ fatty alcohol glucoside, Coco-glucoside (INCI);
10. PLANTACARE® 818 UP/MB* Surfactant—a C₈-C₁₆ fatty alcohol glucoside, Mass Balance, Coco-glucoside (INCI);
11. PLANTACARE® 1200 UP Surfactant—a C₁₂-C₁₆ fatty alcohol glucoside, lauryl glucoside (INCI);
12. PLANTACARE® 1200 UP/MB* Surfactant—a C₁₂-C₁₆ fatty alcohol glucoside, lauryl glucoside (INCI);
13. PLANTACARE® 2000 UP Surfactant—a C₈-C₁₆ fatty alcohol glucoside, decyl glucoside (INCI);
14. PLANTACARE® 2000 UP/MB* Surfactant—a C₈-C₁₆ fatty alcohol glucoside, Mass Balance, decyl glucoside (INCI); and
15. PLANTACARE® 810 UP Surfactant—a C₈-C₁₀ fatty alcohol glucoside, caprylyl/capryl glucoside (INCI).

In an embodiment, the second nonionic surfactant is at least one selected from surfactant having the following general formula (I) or (II):

R(X)ₙOR′ (I)
wherein, R represents a linear or branched C₈-C₃₀ alkyl group or C₈-C₃₀ alkenyl group, preferably a linear or branched C₁₀-C₃₀ alkyl group or C₁₀-C₃₀ alkenyl group, and more preferably a linear or branched C₁₂-C₂₄ alkyl group or C₁₂-C₂₄ alkenyl group;
   X represents —OCH₂CH₂— or —OCH₂—CHCH₃—, and more particularly —OCH₂CH₂—;
   n represents an integer from 2 to 100, and preferably an integer from 3 to 90, such as an integer from 4 to 80, an integer from 5 to 70, an integer from 6 to 60, an integer from 7 to 50, an integer from 8 to 40, an integer from 9 to 30; and
   R′ represents H or a linear or branched C₈-C₃₀ alkyl group or C₈-C₃₀ alkenyl group, preferably H, or a linear or branched C₁₀-C₃₀ alkyl group or C₁₀-C₃₀ alkenyl group, more preferably H, or a linear or branched C₁₂-C₂₄ alkyl group or C₁₂-C₂₄ alkenyl group, and most preferably H;

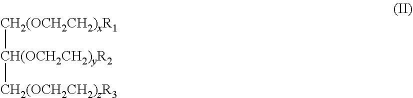

wherein, the sum of x+y+z represents an integer from 2 to 100, and preferably an integer from 3 to 90, such as an integer from 4 to 80, an integer from 5 to 70, an integer from 6 to 60, an integer from 7 to 50, an integer from 8 to 40, an integer from 9 to 30; and
   R₁, R₂, and R₃, are the same or different and each independently represents OH, and/or a linear or branched C₈-C₃₀ fatty acid ester group, preferably OH, and/or a linear or branched C₁₀-C₃₀ fatty acid ester group, or a linear or branched C₁₂-C₂₄ fatty acid ester group, with the proviso that at least one of R₁, R₂ and R₃ is not OH.

In an embodiment, the second nonionic surfactant is at least one selected from surfactant having the following general formula (I) or (II):

R(X)ₙOR′ (I)
wherein, R represents a linear or branched C₁₂-C₂₄ alkyl group or C₁₂-C₂₄ alkenyl group;
   X represents —OCH₂CH₂—;
   n represents an integer from 2 to 30, such as an integer from 3 to 30, an integer from 4 to 30, an integer from 5 to 30, an integer from 6 to 30, an integer from 7 to 30 and an integer from 8 to 30; and
   R′ represents H;

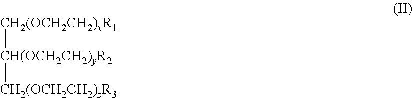

wherein, the sum of x+y+z represents an integer from 2 to 30, such as an integer from 3 to 30, an integer from 4 to 30, an integer from 5 to 30, an integer from 6 to 30, an integer from 7 to 30 and an integer from 8 to 30; and
R₁, R₂, and R₃, are the same or different and each independently represents OH, and/or a linear or branched C₁₂-C₂₄ fatty acid ester group, with the proviso that at least one of R₁, R₂ and R₃ is not OH.

In an embodiment, the second nonionic surfactant comprises at least one having the following general formula (I):

R(X)ₙOR′ (I)
wherein, R represents a linear or branched C₈-C₃₀ alkyl group or C₈-C₃₀ alkenyl group, preferably a linear or branched C₁₀-C₃₀ alkyl group or C₁₀-C₃₀ alkenyl group, and more preferably a linear or branched C₁₂-C₂₄ alkyl group or C₁₂-C₂₄ alkenyl group; X represents —OCH₂CH₂— or —OCH₂—CHCH₃—, and more particularly —OCH₂CH₂—;
   n represents an integer from 2 to 100, and preferably an integer from 3 to 90, such as an integer from 4 to 80, an integer from 5 to 70, an integer from 6 to 60, an integer from 7 to 50, an integer from 8 to 40, an integer from 9 to 30; and
   R′ represents H or a linear or branched C₈-C₃₀ alkyl group or C₈-C₃₀ alkenyl group, preferably H, or a linear or branched C₁₀-C₃₀ alkyl group or C₁₀-C₃₀ alkenyl group, more preferably H, or a linear or branched C₁₂-C₂₄ alkyl group or C₁₂-C₂₄ alkenyl group, and most preferably H.

In an embodiment, the second nonionic surfactant comprises at least one having the following general formula (I):

R(X)ₙOR′ (I)
wherein, R represents a linear or branched C₁₂-C₂₄ alkyl group or C₁₂-C₂₄ alkenyl group;
   X represents —OCH₂CH₂—;
   n represents an integer from 2 to 30, such as an integer from 3 to 30, an integer from 4 to 30, an integer from 5 to 30, an integer from 6 to 30, an integer from 7 to 30 and an integer from 8 to 30; and
   R′ represents H.

In an embodiment, the second nonionic surfactant comprises at least one having the following general formula (II):

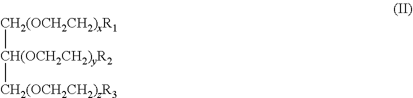

wherein, the sum of x+y+z represents an integer from 2 to 100, and preferably an integer from 3 to 90, such as an integer from 4 to 80, an integer from 5 to 70, an integer from 6 to 60, an integer from 7 to 50, an integer from 8 to 40, an integer from 9 to 30; and
   R₁, R₂, and R₃, are the same or different and each independently, represents OH, and/or a linear or branched C₈-C₃₀ fatty acid ester group, preferably OH, and/or a linear or branched C₁₀-C₃₀ fatty acid ester group, more preferably OH, and/or a linear or branched C₁₂-C₂₄ fatty acid ester group, with the proviso that at least one of R₁, R₂ and R₃ is not OH.

In an embodiment, the second nonionic surfactant comprises at least one having the following general formula (II):

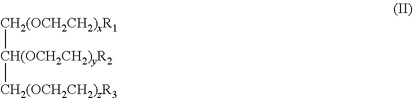

wherein, the sum of x+y+z represents an integer from 2 to 30, such as an integer from 3 to 30, an integer from 4 to 30, an integer from 5 to 30, an integer from 6 to 30, an integer from 7 to 30 and an integer from 8 to 30; and R₁, R₂, and R₃, are the same or different and each independently represents OH, and/or a linear or branched C₁₂-C₂₄ fatty acid ester group, with the proviso that at least one of R₁, R₂ and R₃ is not OH.

In an embodiment, the second nonionic surfactant may comprise at least one selected from the group consisting of polyoxyethylene ethers of lauryl alcohol, macrogol lauryl ether, polyoxyethylene ethers of cetyl alcohol, polyoxyethylene ethers of cetylstearyl alcohol, polyoxyethylene ethers of stearyl alcohol, polyoxyethylene ethers of oleyl alcohol, polyoxyethylene tridecyl ether, polyoxyethylene ethers of glyceryl laurate, polyoxyethylene ethers of glyceryl cocoate, polyoxyethylene ethers of glyceryl isostearate, polyoxyethylene ethers of glyceryl diisostearate, polyoxyethylene ethers of glyceryl oleate, polyoxyethylene ethers of glyceryl ricinoleate, polyoxyethylene ethers of glyceryl stearate, polyoxyethylene ethers of glyceryl triisostearate, polyoxyethylene ethers of glyceryl trioleate, polyoxyethylene ethers of glyceryl tristearate, and a combination thereof.

In an embodiment, the second nonionic surfactant may comprise at least one selected from the group consisting of polyoxyethylene ethers of lauryl alcohol, macrogol lauryl ether, polyoxyethylene ethers of cetyl alcohol, polyoxyethylene ethers of cetylstearyl alcohol, polyoxyethylene ethers of stearyl alcohol, polyoxyethylene ethers of oleyl alcohol, polyoxyethylene tridecyl ether, and a combination thereof.

In an embodiment, the second nonionic surfactant may comprise at least one selected from the group consisting of polyoxyethylene ethers of glyceryl laurate, polyoxyethylene ethers of glyceryl cocoate, polyoxyethylene ethers of glyceryl isostearate, polyoxyethylene ethers of glyceryl diisostearate, polyoxyethylene ethers of glyceryl oleate, polyoxyethylene ethers of glyceryl ricinoleate, polyoxyethylene ethers of glyceryl stearate, polyoxyethylene ethers of glyceryl triisostearate, polyoxyethylene ethers of glyceryl trioleate, polyoxyethylene ethers of glyceryl tristearate, and a combination thereof.

In an embodiment, the second nonionic surfactant may comprise at least one of the following:
polyoxyethylene (4) lauryl ether (Laureth-4, INCI);
polyoxyethylene(9) lauryl ether (Laureth-9, INCI);
polyoxyethylene (23) lauryl ether (Laureth-23, INCI);
polyoxyethylene (2) cetyl ether (Ceteth-2, INCI);
polyoxyethylene (10) cetyl ether (Ceteth-10, INCI);
polyoxyethylene (15) cetyl ether (Ceteth-15, INCI);
polyoxyethylene (20) cetyl ether (Ceteth-20, INCI);
polyoxyethylene (30) cetyl ether (Ceteth-30, INCI);
polyoxyethylene (6) cetylstearyl ether (Ceteareth-6, INCI);
polyoxyethylene (12) cetylstearyl ether (Ceteareth-12, INCI);
polyoxyethylene (20) cetylstearyl ether (Ceteareth-20, INCI);
polyoxyethylene (25) cetylstearyl ether (Ceteareth-25, INCI);
polyoxyethylene (30) cetylstearyl ether (Ceteareth-30, INCI);
polyoxyethylene (2) stearyl ether (Steareth-2, INCI);
polyoxyethylene (10) stearyl ether (Steareth-10, INCI);
polyoxyethylene (20) stearyl ether (Steareth-20, INCI);
polyoxyethylene (21) stearyl ether (Steareth-21, INCI);
polyoxyethylene (30) stearyl ether (Steareth-30, INCI);
polyoxyethylene (2) oleyl ether (Oleth-2, INCI);
polyoxyethylene (10) oleyl ether (Oleth-10, INCI);
polyoxyethylene (15) oleyl ether (Oleth-15, INCI);
polyoxyethylene (20) oleyl ether (Oleth-20, INCI);
polyoxyethylene (23) oleyl ether (Oleth-23, INCI);
polyoxyethylene (30) oleyl ether (Oleth-30, INCI);
polyoxyethylene (25) octyldodecyl ether;
polyoxyethylene (25) decyltetradecyl ether;
polyoxyethylene (9) C₁₂₋₁₄ secondary alkyl ether;
polyoxyethylene (12) C₁₂₋₁₄ secondary alkyl ether;
polyoxyethylene (20) behenyl ether;
polyoxyethylene (25) behenyl ether (Beheneth-25, INCI);
polyoxyethylene (30) behenyl ether; and
polyoxyethylene (10) tridecyl ether (Trideceth-10, INCI);
polyoxyethylene (12) ether of glyceryl laurate;
polyoxyethylene (20) ether of glyceryl laurate;
polyoxyethylene (30) ether of glyceryl laurate;
polyoxyethylene (20) ether of glyceryl cocoate;
polyoxyethylene (30) ether of glyceryl cocoate;
polyoxyethylene (10) ether of glyceryl isostearate;
polyoxyethylene (20) ether of glyceryl isostearate;
polyoxyethylene (30) ether of glyceryl isostearate;
polyoxyethylene (10) ether of glyceryl diisostearate;
polyoxyethylene (20) ether of glyceryl diisostearate;
polyoxyethylene (30) ether of glyceryl diisostearate;
polyoxyethylene (10) ether of glyceryl oleate;
polyoxyethylene (20) ether of glyceryl oleate;
polyoxyethylene (30) ether of glyceryl oleate;
polyoxyethylene (15) ether of glyceryl ricinoleate;
polyoxyethylene (20) ether of glyceryl ricinoleate;
polyoxyethylene (10) ether of glyceryl stearate;
polyoxyethylene (20) ether of glyceryl stearate;
polyoxyethylene (30) ether of glyceryl stearate;
polyoxyethylene (10) ether of glyceryl triisostearate;
polyoxyethylene (20) ether of glyceryl triisostearate;
polyoxyethylene (30) ether of glyceryl triisostearate;
polyoxyethylene (10) ether of glyceryl trioleate;
polyoxyethylene (20) ether of glyceryl trioleate;
polyoxyethylene (30) ether of glyceryl trioleate;
polyoxyethylene (10) ether of glyceryl tristearate;
polyoxyethylene (20) ether of glyceryl tristearate.

Preferably, the second nonionic surfactant is selected from the group consisting of polyoxyethylene (23) lauryl ether (Laureth-23, INCI), polyoxyethylene (20) cetyl ether (Ceteth-20, INCI), polyoxyethylene (30) cetyl ether (Ceteth-30, INCI), polyoxyethylene (12) cetylstearyl ether (Ceteareth-12, INCI); polyoxyethylene (20) cetylstearyl ether (Ceteareth-20, INCI), polyoxyethylene (25) cetylstearyl ether (Ceteareth-25, INCI); polyoxyethylene (30) cetylstearyl ether (Ceteareth-30, INCI); polyoxyethylene (20) stearyl ether (Steareth-20, INCI), polyoxyethylene (21) stearyl ether (Steareth-21, INCI); polyoxyethylene (30) stearyl ether (Steareth-30, INCI), polyoxyethylene (15) oleyl ether (Oleth-15, INCI); polyoxyethylene (20) oleyl ether (Oleth-20, INCI), polyoxyethylene (23) oleyl ether (Oleth-23, INCI), polyoxyethylene (30) oleyl ether (Oleth-30, INCI) polyoxyethylene (20) behenyl ether (Beheneth-20, INCI), polyoxyethylene (25) behenyl ether (Beheneth-25, INCI) and polyoxyethylene (30) behenyl ether (Beheneth-30, INCI), polyoxyethylene (20) ether of glyceryl laurate (PEG-20 glyceryl laurate, INCI), polyoxyethylene (20) ether of glyceryl cocoate (PEG-20 glyceryl cocoate, INCI), polyoxyethylene (20) ether of glyceryl isostearate (PEG-20 glyceryl isostearate, INCI), polyoxyethylene (20) ether of glyceryl diisostearate (PEG-20 glyceryl diisostearate, INCI), polyoxyethylene (20) ether of glyceryl oleate (PEG-20 glyceryl oleate, INCI), polyoxyethylene (20) ether of glyceryl ricinoleate (PEG-20 glyceryl ricinoleate, INCI), polyoxyethylene (20) ether of glyceryl stearate (PEG-20 glyceryl stearate, INCI), polyoxyethylene (20) ether of glyceryl triisostearate (PEG-20 glyceryl triisostearate, INCI), polyoxyethylene (20) ether of glyceryl trioleate (PEG-20 glyceryl trioleate, INCI), polyoxyethylene (20) ether of glyceryl tristearate (PEG-20 glyceryl tristearate, INCI) and a combination thereof.

In an embodiment, the nonionic surfactant of general formula (I) may be commercially available, for example as EUMULGIN® 020S, EUMULGIN® BA-25, or EUMULGIN® B3, EUMULGIN® B1, EUMULGIN® B2, EUMULGIN® B25, EUMULGIN® 030 from BASF, Brij® 35, Brij® 52, Brij® 56, Brij® 93, Brij® 97, Brij® 99, Ethylan® 256, Ethylan® 257, Ethylan® 2512, Renex® 30, Renex® 31, Texofor AP, Texofor A6, Texofor A10, Emalex GM-20, Emalex GWS-320, Emalex GWIS-120, Emalex GWIS-220EX, Emalex GWIS-320, Emalex GWO-320, Uniox GT-201S or mixtures thereof.

In an embodiment, the aqueous phase of the cosmetic composition comprises water, such as purified water, deionized water or floral water, and optionally one or more water miscible solvents. According to any one of the inventive embodiments, the aqueous phase may be any cosmetically acceptable water based materials, such as deionized water, purified water or floral water.

In an embodiment, the C₂-C₆ polyol is one or more selected from the group consisting of glycerin, propanediol, butanediol, and sorbitol. The butanediol may be 1,3-butanediol. In an embodiment, the C₂-C₆ polyol is glycerin.

According to any one of the inventive embodiments, the oil phase may comprise one or more cosmetically or acceptable oils or mixtures thereof.

The oil may be selected from the group consisting of oils of animal or plant origin, mineral oils, synthetic glycerides, fatty esters, fatty alcohols, silicone oils and aliphatic hydrocarbons. These materials may be volatile or non-volatile. Volatile oils may be used in combination with non-volatile oils and/or other wax mentioned in the present description. Suitable oil may be selected from aliphatic hydrocarbons, plant oils, fatty alcohols, esters of fatty alcohols and/or fatty acids other than animal or plant oils and synthetic glycerides, or mixtures thereof. Particularly suitable oil may be selected from the group consisting of plant oils, silicone oils, esters of fatty alcohols, and mixtures thereof.

Suitable plant oils for use in the cosmetic composition of the invention may nonexclusively include linseed oil, camellia oil, sunflower oil, apricot oil, hazelnut oil, vegetable squalane oil, sasanqua oil, grapeseed oil, peanut oil, coconut oil, palm kernel oil, soybean oil, macadamia nut oil, avocado oil, safflower oil, sweet almond oil, apricot oil, corn oil, jojoba oil, olive oil, sesame oil, palm oil, eucalyptus oil, rosemary oil, lavender oil, pine oil, thyme oil, mint oil, cardamom oil, orange-blossom oil, bran oil, rice oil, rapeseed oil, castor oil, and mixtures thereof.

Suitable animal oils for use in the cosmetic composition of the invention may nonexclusively include squalene, perhydrosqualene, squalane and mixtures thereof.

Silicone oils for use in the cosmetic composition of the invention either may be volatile and/or non-volatile silicon oils. Preferred silicone oils are non-volatile silicon oils known with their INCI name as dimethicone and dimethiconol. Volatile silicone oils such as cyclomethicones may be used in combination with non-volatile silicones and/or other wax and/or oils mentioned in the present description. As examples of silicone oils, mention can be made of, for example, linear organopolysiloxanes such as diemethylpolysiloxane, methylphenylpolysiloxane, methylhydrogenpolysiloxane, and the like; cyclic organopolysiloxanes such as cyclopentasiloxane, cyclohexylsiloxane, octamethylcyclotetrasiloxane, decamethylcyclopentasiloxane, dodecamethylcyclohexasiloxane, and mixtures thereof. Furthermore, arylated silicones comprising at least one aryl group in its molecule such as phenyl methiocone, phenyl trimethicone, diphenyl dimethicone, diphenylsiloxy phenyl trimethicone, tetramethyl tetraphenyl trisiloxiane, triphenyl trimethicone, tetramethyl tetraphenyl trisiloxane and trimethyl pentaphenyl trisiloxane can be comprised in the compositions of the invention.

Suitable fatty esters or esters of fatty alcohols for use in the cosmetic composition of the invention may nonexclusively include triglycerides such as triethylhexanoin and caprylic capric triglyceride, dioctyl carbonate, diisopropyl sebacate, ethyl laurate, butyl laurate, hexyl laurate, isohexyl laurate, isopropyl laurate, methyl myristate, ethyl myristate, butyl myristate, isobutyl myristate, isopropyl myritate, 2-octyldodecyl myristate, 2-ethylhexyl monococoate (or octyl monococoate), ethyl palmitate, isopropyl palmitate, isobutyl palmitate, 2-ethylhexyl palmitate (or octyl palmitate), butyl stearate, isopropyl stearate, isobutyl stearate, isocetyl stearate, isosteary isostearate, isopropyl isostearate, 2-ethylhexyl stearate (or octyl stearate), decyl oleate, isononyl isononanoate, tridecyl neopentanoate, isocetyl neopentanoate, isostearyl neopentanoate, octyldodecyl neopentanoate and isoarachidyl neopentanoate, and mixtures thereof.

In an embodiment, the suitable oils may be commercially available, for example as MYRITOL® GTEH-SD, MYRITOL® 318 RC, CETIOL® CC, CEGESOFT® PS6, and CEGESOFT® C24 RC, CETIOL® ININ, CETIOL® SN-1 SD, CETIOL® ULTIMATE. All commercially available cosmetically acceptable oils or mixtures thereof are suitable for use in the compositions of the invention.

In an embodiment, the cosmetic composition may be free or substantially free of polymeric thickener, gelling agent or thickening polymer. For the purpose of the present invention, the expression “substantial free of” means that the amount of the polymeric thickener in the composition is lower than the amount conventionally used in the art (conventionally in the range of from 5 wt % to 20 wt % based on the total weight of the cosmetic composition) for the purpose of thickening, such that it does not show significant thickening effect on the composition that can be observed or measured. More particularly, the amount of the polymeric thickener or gelling agent or thickening polymer, if present, may be present in an amount of lower than 5 wt % of the cosmetic composition, such as in an amount of lower than 4 wt %, lower than 3 wt %, lower than 2 wt %, lower than 1 wt %, lower than 0.8 wt %, lower than 0.6 wt %, lower than 0.5 wt %, lower than 0.4 wt %, lower than 0.3 wt %, lower than 0.2 wt % or lower than 0.1 wt % of the cosmetic composition.

In an embodiment, the cosmetic composition may further comprise one or more surfactants selected from the group consisting of anionic surfactant, amphoteric surfactant and cationic surfactant, and mixtures thereof, as long as they are compatible with the above first and second non-ionic surfactants. In an embodiment, based on the total weight of the cosmetic composition, the one or more surfactants selected from the group consisting of anionic surfactant, amphoteric surfactant and cationic surfactant, and mixtures thereof may be present in an amount of from 0.1 wt % to 15 wt %, such as from 0.2 wt % to 14 wt %, from 0.3 wt % to 13 wt %, from 0.4 wt % to 12 wt %, from 0.5 wt % to 11 wt %, from 0.6 wt % to 10 wt %, from 0.7 wt % to 9 wt %, from 0.8 wt % to 8 wt %, from 0.9 wt % to 9 wt %, from 1 wt % to 8 wt %, from 1.1 wt % to 7 wt %, from 1.2 wt % to 6.5 wt %, from 1.3 wt % to 6 wt %, from 1.4 wt % to 5.5 wt %, from 1.5 wt % to 5 wt %, from 1.6 wt % to 4.5 wt %, from 1.7 wt % to 4 wt %, from 1.8 wt % to 3.5 wt %, from 1.9 wt % to 3 wt %.

The compatible anionic surfactants may nonexclusively include alkyl sulphates, alkyl ether sulphates, alkaryl sulphonates, alkanoyl isethionates, alkyl succinates, alkyl sulphosuccinates, N-alkoyl sarcosinates, alkyl phosphates, alkyl ether phosphates, alkyl ether carboxylates, and alpha-olefin sulphonates, especially their sodium, magnesium ammonium and mono-, di- and tri-thanolamine salts.

The compatible anionic surfactants may be selected from the group consisting of sodium lauryl sulphate, triethanolamine lauryl sulphate, triethanolamine monlauryl phosphate, sodium lauryl ether sulphate 1 EO, 2EO and 3EO, ammonium lauryl sulphate and ammonium lauryl ether sulphate 1EO, 2EO and 3EO.

The compatible amphoteric surfactants may nonexclusively include alkyl amine oxides, alkyl betaines, alkyl amidopropyl betaines, alkyl sulphobetaines (sultaines), alkyl glycinates, alkyl carboxyglycinates, alkyl amphopropionates, alkyl amphoglycinates, alkyl amidopropyl hydroxysultaines, acyl taurates and acyl glutamates, wherein the alkyl and acyl groups have from 8 to 19 carbon atoms. Examples include lauryl amine oxide, cocodimethyl sulphopropyl betaine, lauryl betaine, cocamidopropyl betaine and sodium cocamphopropionate.

The compatible cationic surfactants may nonexclusively include quaternary ammonium hydroxides, e.g., tetramethylammonium hydroxide, alkyltrimethylammonium hydroxides wherein the alkyl group has from about 8 to 22 carbon atoms, for example octyltrimethylammonium hydroxide, dodecyltrimethylammonium hydroxide, hexadecyltrimethylammonium hydroxide, cetyltrimethylammonium hydroxide, octyldimethylbenzylammounium hydroxide, decyldimethyl-benzylammonium hydroxide, dioctadecyldimethylammonium hydroxide, tallow trimethylammonium hydroxide, cocotrimetylammonium hydroxide and the corresponding salts thereof containing anions other than hydroxide, e.g., chlorides, cetylpyridinium hydroxide or salts thereof (e.g., chloride), Quaternium-5, Quaternium-31, Quaternium-18, and mixtures thereof.

The cosmetic composition of the present invention may comprise, besides the above-mentioned components, additives generally blended in a cosmetic composition such as moisturizers, antiwrinkle/antiaging agents, cellular stimulants, anti-inflammatory agents, antioxidants, UV absorption/scattering agents, preservatives, pH adjusters, colorants, flavors and the like, as long as the characteristics of the cosmetic composition of the invention are not impaired.

Suitable moisturizers for use in the cosmetic composition of the invention may nonexclusively include polyvalent alcohol such as glycerol, 1,3-propanediol, sorbitol and the like; mucopolysaccharides such as sodium hyaluronate, chondroitin sulfate and the like; amino acids such as alanine, sodium pyrrolidonecarboxylate and the like or a salt thereof.

Suitable antiwrinkle/antiaging agents for use in the cosmetic composition of the invention may nonexclusively include hydrolyzed eggshell membrane, atelocollagen, rice bran extract, rooibos extract and the like.

Suitable cellular stimulants for use in the cosmetic composition of the invention may nonexclusively include sodium salt of deoxyribonucleic acid, yeast extract, Asian ginseng extract and the like.

Suitable anti-inflammatory agents for use in the cosmetic composition of the invention may nonexclusively include allantoin, aloe vera extract, krantz aloe extract, chamomile extract, licorice extract, dipotassium glycyrrhizate and the like.

Suitable antioxidants for use in the cosmetic composition of the invention may nonexclusively include vitamin E such as tocopherol acetate, d-δ-tocopherol, dl-α-tocopherol, natural vitamin E and the like; polyphenols such as glucosylrutin, tannic acid and the like; gallic acids such as gallic acid, propyl gallate and the like and a derivative thereof; plant extracts such as Japanese basil leaf extract, sage leaf extract and the like.

Suitable UV absorption/scattering agents for use in the cosmetic composition of the invention may nonexclusively include methylene bis-benzotriazolyl tetramethylbutylphenol, bis-ethylhexyloxyphenol methoxyphenyl triazine, diethylamino hydroxybenzoyl hexyl benzoate, paradimethylaminobenzoate 2-ethylhexyl, oxybenzone-3-(2-hydroxy-4-methoxybenzophenone), paramethoxycinnamic acid 2-ethylhexyl, 4-tert-butyl-4′-methoxydibenzoylmethane, titanium oxide and the like.

Suitable preservatives for use in the cosmetic composition of the invention may nonexclusively include sodium benzoate, phenoxyethanol, paraoxybenzoates such as methyl p-hydroxybenzoate, ethyl parahydroxybenzoate, propyl p-hydroxybenzoate and the like.

Suitable pH adjusters for use in the cosmetic composition of the invention may nonexclusively include succinic acid, citric acid, sodium citrate, tartaric acid, sodium tartarate, sodium hydroxide, potassium hydroxide, triethanolamine, gluconolactone and the like.

Suitable colorants for use in the cosmetic composition of the invention may nonexclusively include inorganic pigments such as iron blue, ultramarine blue, red iron oxide, black iron oxide, yellow iron oxide, talc, kaolin, manganese violet, carbon black and the like; natural dyes such as β-carotene, lycopene, shisonin, safflor yellow, shikonin, chlorophyll and the like, tar pigments such as red No. 102, red No. 201, Blue No. 202 and the like, lake pigments such as red No. 3 aluminum lake, yellow No. 4 aluminum lake, blue No. 1 barium lake and the like.

Suitable flavors for use in the cosmetic composition of the invention may nonexclusively include natural flavors such as cinnamon oil, lavender oil, jasmine oil, peppermint oil, orange oil, rose oil and the like; synthetic flavors such as citronellol, eugenol, geraniol, menthol and the like.

The composition of the invention may also include other additives such as, but not limited to, abrasives, absorbents, a foam building agent, antifoaming agents, antimicrobial agents (e.g., iodopropyl butylcarbamate), biological additives, bulking agents, chelating agents, film formers or materials, e.g., polymers for aiding the film-forming properties of the composition (e.g., copolymer of eicosene and vinyl pyrrolidone), propellants, reducing agents, skin bleaching and lightening agents (e.g., hydroquinone, kojic acid, ascorbic acid, magnesium ascorbyl phosphate, ascorbyl glucosamine), skin soothing and/or healing agents (e.g., panthenol and derivatives (e.g., ethyl panthenol), aloe vera, pantothenic acid and its derivatives, allantoin, bisabolol, silicones, and fatty alcohols.

One or more kinds of the above-mentioned additives can be present in the cosmetic composition of the invention in amounts generally used for cosmetic compositions, as long as the characteristics of the cosmetic composition of the invention are not impaired.

In a second aspect, the invention provides a method for preparing the cosmetic composition of the invention, comprising the following steps:
(a) adding the surfactant portion, the C₂-C₆ polyol to the aqueous phase, and mixing—optionally and preferably with heating, stirring or simultaneous heating and stirring—them uniformly;
(b) heating the oil phase;
(c) adding the oil phase obtained in step (b) into the aqueous phase obtained in step (a) under stirring; and
(d) cooling the mixture obtained in step (c) under stirring to give a gel.

In an embodiment, the aqueous mixture in step (a) may be heated to a temperature in the range of 50° C.-80° C., preferably in the range of 55° C.-75° C., more preferably in the range of 60° C.-70° C. and most preferably in the range of 65° C.-70° C.

In an embodiment, the oil phase in step (b) may be heated to a temperature in the range of 50° C.-80° C., preferably in the range of 55° C.-75° C., more preferably in the range of 60° C.-70° C. and most preferably in the range of 65° C.-70° C.

In an embodiment, in step (c), the adding rate of the oil phase into the aqueous phase may be in the range of from 1 wt % to 8 wt % of the oil phase per minute, such as from 2 wt % to 6 wt %, from 2 wt % to 5 wt %, and from 3 wt % to 5 wt % of the oil phase per minute. The adding rate through the whole adding process may vary depending on the actual situation, while ensuring the uniformly emulsifying. For example, during the early period, the adding rate may be 2 wt % of the oil phase per minute, and during the middle and later periods, the adding rate may be 3 wt % to 5 wt % of the oil phase per minute.

In an embodiment, the stirring of step (c) may be carried out at 200-400 rpm.

In an embodiment, the mixture obtained in step (c) may be cooled to the ambient temperature to give a gel.

In a third aspect, the invention provides use of the cosmetic composition for the preparation of personal care products.

In an embodiment, the personal care products may be one of the following: skin care 30 products, baby care products, hair care products, cleansing products, makeup removers, massage products, bath products, shaving products, cosmetics, tooth pastes, deodorants, anti-perspirants, insect repellants, shampoos, hair conditioners, sun care products, shower gels, hair styling gels, hair anti-dandruff products, hair growth promoter products, hair colorant products, hair bleaching agent products, hair anti-frizzing agent products, hair relaxer products, lubricating gel products, and spermicide gel products.

In a fourth aspect, the invention provides a method for personal care or cleansing, comprising the step of applying the cosmetic composition of the invention to a desired location on the skin or hair in the presence of water, and then rinsing off the composition from the skin or hair with water.

In an embodiment, after being applied to the skin or hair, the cosmetic composition of the invention produces significant amount of cleansing foams.

In an embodiment, the method is a facial cleansing method and preferably a make-up removing method, and the composition is applied to at least one part of the face.

For the purposes of the present invention, the term “personal care” is intended to refer to cosmetic and skin care compositions for application to the skin, including, for example, makeup removers, body washes and cleansers, as well as leave-on application to the skin. In the present invention, the term “personal care” is also intended to refer to hair care compositions including, for example, shampoos, leave-on conditioners, rinse-off conditioners, styling gels, pomades, hair coloring products (e.g., two-part hair dyes), hairsprays, and mousses. Preferably, the personal care composition is cosmetically acceptable. “Cosmetically acceptable” is intended to underscore that materials that are toxic when present in the amounts typically found in personal care compositions are not contemplated as part of the present disclosure.

For the purposes of the present invention, the term “semi-transparent” means that the cosmetic composition of the invention allows light to partially pass through it without however making it possible to clearly distinguish an object on the other side.

The aspects of the present invention will become apparent from the specific examples below. It should be understood that the specific examples, while indicating the preferred embodiment of the invention, are intended for purposes of illustration only and are not intended to limit the scope of the invention.

### EXAMPLES

Some embodiments of the invention will be described in detail in the following Examples.

### Example 1: Formulations of the Gels of the Invention

Following Formulations 1 to 3 as shown in Table 1 were prepared.

TABLE 1

| | Ingredient | | | |
|---|---|---|---|---|
| | (INCI name | Formulation | Formulation | Formulation |
| | (Trade name)) | 1 (wt %) | 2 (wt %) | 3 (wt %) |
| Surfactant | Oleth-20 | 3 | 0 | 6 |
| portion | (EUMULGIN ^{®} | | | |
| | O 20 S) | | | |
| | Lauryl glucoside | 6 | 12 | 0 |
| | (PLANTACARE ^{®} | | | |
| | 1200 UP (50% | | | |
| | active in | | | |
| | purified water)) | | | |
| Polyol | Glycerin | 6 | 6 | 6 |
| Aqueous | Purified Water | 3 | 0 | 6 |
| phase | | | | |
| Oil phase | Triethylhexanoin | 82 | 82 | 82 |
| | (MYRITOL ® | | | |
| | GTEH) | | | |

Formulation 1 was prepared according to the following method:
(a) adding the surfactant portion—Oleth-20 and Lauryl glucoside, the glycerin to the purified water, heating to a temperature of 65° C., stirring and mixing them uniformly;
(b) heating the oil phase to a temperature of 65° C.;
(c) adding the oil phase obtained in step (b) into the aqueous phase obtained in step (a) under stirring at 300 rpm over 30 minutes, while controlling the adding rate of the oil phase in the range of 2 wt % to 5 wt % of the oil phase per minute; and
(d) cooling the mixture obtained in step (c) under stirring to ambient temperature, and thus giving a gel.

Formulations 2 and 3 were prepared according to the above method for preparing Formulation 1. In Formulation 2, as the PLANTACARE® 1200 UP already contained 50% of purified water, which formed the aqueous phase, no additional purified water was added. Thus, Formulation 2 had 6 wt % of purified water based on the total weight of the formulation.

Following Formulations 4 to 6 as shown in Table 2 were prepared according to the above method for preparing Formulation 1.

TABLE 2

| | Ingredient | | | |
|---|---|---|---|---|
| | (INCI name | Formulation | Formulation | Formulation |
| | (Trade name)) | 4 (wt %) | 5 (wt %) | 6 (wt %) |
| Surfactant | Oleth-20 | 3 | 6 | 0 |
| portion | (EUMULGIN ^{®} | | | |
| | O 20 S) | | | |
| | Coco-glucoside | 6 | 0 | 12 |
| | (PLANTACARE ^{®} | | | |
| | 818 UP (50% | | | |
| | active in purified | | | |
| | water)) | | | |
| Polyol | Glycerin | 6 | 6 | 6 |
| Aqueous | Purified Water | 3 | 6 | 0 |
| phase | | | | |
| Oil phase | Caprylic capric | 82 | 82 | 82 |
| | triglyceride | | | |
| | (MYRITOL ^{®} | | | |
| | 318 RC) | | | |

Following Formulations 7 to 10 having different oils as shown in Table 3 were prepared according to the above method for preparing Formulation 1.

TABLE 3

| | Ingredient | | | | |
|---|---|---|---|---|---|
| | (INCI name | For- | For- | For- | For- |
| | and/or Trade | mulation | mulation | mulation | mulation |
| | name)) | 7 (wt %) | 8 (wt %) | 9 (wt %) | 10 (wt %) |
| Surfactant | Lauryl | 7 | 7 | 7 | 7 |
| portion | glucoside | | | | |
| | (PLANTACAR | | | | |
| | E ^{®} 1200 UP | | | | |
| | (50% active in | | | | |
| | purified | | | | |
| | water)) | | | | |
| | Oleth-20 | 4 | 4 | 4 | 4 |
| | (EUMULGIN ^{®} | | | | |
| | O 20 S) | | | | |
| Aqueous | Purified Water | 4 | 4 | 4 | 4 |
| phase | | | | | |
| Polyol | Glycerin | 7.5 | 7.5 | 7.5 | 7.5 |
| | Undecane | | | | |
| | (and) | | | | |
| | Tridecane | 77.5 | | | |
| | (CETIOL ^{®} | | | | |
| | ULTIMATE) | | | | |
| | Dicaprylyl | | 77.5 | | |
| | Carbonate | | | | |
| | (CETIOL ^{®} CC) | | | | |
| Oil | Triethylhexano | | | 77.5 | |
| phase | in | | | | |
| | (MYRITOL ® | | | | |
| | GTEH) | | | | |
| | Ethylhexyl | | | | 77.5 |
| | Palmitate | | | | |
| | (CEGESOFT ^{®} | | | | |
| | C 24 RC) | | | | |

Following formulations 11 to 15 as shown in Table 4.1 were prepared according to the above method for preparing Formulation 1.

TABLE 4.1

| | Ingredient | Formulation | Formulation | Formulation | Formulation | Formulation |
|---|---|---|---|---|---|---|
| | (INCI name and/or Trade | 11 (wt %) | 12 (wt %) | 13 (wt %) | 14 (wt %) | 15 (wt %) |
| | name)) | (2:8)* | (3:7)* | (4:6)* | (6:4)* | (7:3)* |
| | Lauryl glucoside | 2.4 | 3.6 | 4.8 | 7.2 | 8.4 |
| Surfactant | (PLANTACARE ® 1200 UP (50% | | | | | |
| potion | active in purified water)) | 4.8 | 4.2 | 3.6 | 2.4 | 1.8 |
| | Oleth-20 | | | | | |
| | (EUMULGIN ®O 20 S) | | | | | |
| Polyol | Glycerin | 6 | 6 | 6 | 6 | 6 |
| Aqueous | Purified Water | 4.8 | 4.2 | 3.6 | 2.4 | 1.8 |
| phase | Triethylhexanoin | 82 | 82 | 82 | 82 | 82 |
| Oil phase | (MYRITOL ® GTEH) | | | | | |

Note:
( )* indicates the weight ratio of the first non-ionic surfactant and the second non-ionic surfactant in the surfactant portion. For example, (2:8)* means that Formulation 11 comprises the first and second non-ionic surfactants at weight ratio of 2:8.
As the PLANTACARE ® 1200 UP and 818 UP used in the formulations already contained 50% of purified water, the aqueous phase of the formulation comprised the purified water that was contained in PLANTACARE ® 1200 UP and 818 UP and the portion that was added according to the amount as shown under the item of “aqueous phase” in the tables. For example, Formulations 2, 4 and 6 actually had 6 wt % of purified water based on the total weight of the formulation.

Following formulations 16 to 20 as shown in Table 4.2 were prepared according to the above method for preparing Formulation 1, except that PEG-20 Glyceryl stearate was used as the second non-ionic surfactant. Formulation 21 as shown in Table 4.2 was prepared according to the above method for preparing Formulation 1 except that the surfactant portion contains only PEG-20 Glyceryl stearate.

TABLE 4.2

| | Ingredient | Formulation | Formulation | Formulation | Formulation | Formulation | |
|---|---|---|---|---|---|---|---|
| | (INCI name and/or Trade | 16 (wt %) | 17 (wt %) | 18 (wt %) | 19 (wt %) | 20 (wt %) | Formulation |
| | name)) | (4:6)* | (5:5)* | (6:4)* | (7:3)* | (8:2)* | 21 (wt %) |
| Surfactant | Lauryl glucoside | 2.4 | 3.0 | 3.6 | 4.2 | 4.8 | 0 |
| potion | (PLANTACARE ® 1200 UP | | | | | | |
| | (50% active in purified | | | | | | |
| | water)) | | | | | | |
| | PEG-20 Glyceryl stearate | 3.6 | 3.0 | 2.4 | 1.8 | 1.2 | 6 |
| | (Cutina ® E 24) | | | | | | |
| Polyol | Glycerin | 6 | 6 | 6 | 6 | 6 | 6 |
| Aqueous | Purified Water | 3.6 | 3.0 | 2.4 | 1.8 | 1.2 | 6 |
| phase | | | | | | | |
| Oil phase | Triethylhexanoin | 82 | 82 | 82 | 82 | 82 | 82 |
| | (MYRITOL ® GTEH) | | | | | | |

Note:
( )* indicates the weight ratio of the first non-ionic surfactant and the second non-ionic surfactant in the surfactant portion. For example, (4:6)* means that Formulation 16 comprises the first and second non-ionic surfactants at weight ratio of 4:6.

TABLE 4.2 shows the formulations which use the combination of alkyl polyglucosides and polyoxyethylene fatty acid glyceryl esters. Here, PLANTACARE 1200 UP and Cutina E 24 were used as example formulation. Formulation 21 contains only polyoxyethylene fatty acid glyceryl esters and is used as a comparative example.

### Example 2: Evaluation of the Formulation Stability of Example 1

The above formulations of Example 1 were evaluated for the following aspects immediately after preparation and after storage under different storage conditions.

### 1. Appearance Evaluation

The formulations of Example 1 were observed for the appearance immediately after preparation and after storage at ambient temperature (25° C.) and elevated temperature (50° C.) as shown in the following methods A and B respectively:

Method A: stored under ambient temperature (25° C.) for 3 months; and

Method B: stored under elevated temperature (50° C.) for 1 month.

### 1.1 Transparence

The transparence of the formulations was evaluated according to the following criteria.

Evaluation criteria:
∘: transparent;
Δ: somewhat clouded; and
▪: remarkably clouded.

### 1.2 Color Changing

The presence or absence and the level of color changing was observed and evaluated according to the following criteria. Color changing means the transparency of the sample was decreased when comparing to the initial one.

Evaluation criteria:
∘: color changing is not observed;
Δ: color changing is slightly observed; and
▪: color changing is remarkably observed.

### 1.3 Separation

The presence or absence and the level of separation of oil phase and aqueous phase were observed and evaluated according to the following criteria.

Evaluation criteria:
∘: separation is not observed;
Δ: separation is slightly observed; and
▪: separation is remarkably observed.

### 2. Viscosity Measurement

The viscosity in mPas was determined by rotational method and obtained at 25° C. using a Brookfield viscometer RVDV-II+P (spindle #7, rotational speed as 10 rpm).

### 3. Freeze-Thaw Test

The formulations of Example 1 were also subject to freeze-thaw cycles. One freeze-thaw cycle was processed as follows: first placing the formulation at 25° C. for 24 hours, second at −18° C. for 48 hours, then at 25° C. for 24 hours, and last at 45° C. for 48 hours. After each cycle, the formulations were observed for the appearance, especially for the separation.

### 4. Make-Up Cleansing Test

A make-up removal in-vitro test was conducted as follows:

PMMA board with coarse surface was used to mimic human skin surface. After both sides of the board were cleaned with ethanol and get dried^{\textcircled{1}}, 100 mg of each makeup products (lip stick: M.A.C Lipstick Ruby Woo; foundation: L'oreal Paris foundation Infallible stay fresh 24 h was applied in circles with a diameter of 7 cm on the coarse surface of PMMA board, the makeup products were evenly distributed by finger with glove. After waiting for 30 min^{\textcircled{2}}, 200 ml of makeup remover product was evenly applied on each circle with makeup products, the products were evenly distributed by finger with glove, each area was distributed for 65 circles. Finally, added 2.00 g deionized water onto cotton pads, and used a draw down device to push the wet cotton pads passing through the test areas for twice to remove the residue^{\textcircled{3}}. Pictures were taken in a standardized demo chamber on the three time points: ^{\textcircled{1}}Before applying makeup products (T0); ^{\textcircled{2}}Before applying makeup remover products (T1); ^{\textcircled{3}}After using draw down device to remove the residue (T2). Software was used to assess the grey level for each time points, and calculate make-up removal efficacy % as the result with the formula: Removal efficacy (%)=(T2−T1)/(T0−T1)×100%.

### 5. In-Use Sensory Test

A makeup remover expert panel sensory test was conducted as follows:

Each makeup remover prototypes were evaluated by 12 well-trained panelists with 0-15 rating scale; the assessment mainly focused on product sensory aspects. There 40 were 13 sensory parameters in total, including 3 test processes: before use, in use, and after use. Firstly, appearance and texture (including transparency, and pick up) of the formulation were assessed by visual perception and in before use stage. Then in-use parameters (including speed of oil out, and whiteness as emulsification, etc.) were assessed on the palms and half-face of the panelists. Then, wet skin parameters (including slippery, stickiness) were assessed. Finally, most parameters were assessed in the after-use stage: tissue was used to dry skin by slightly pat, and then instant after dry and 2 min after dry, parameters (including smoothness, tightness, and dryness) were assessed in the defined time points.

The panelists were asked to give product aesthetics scores on these formulations on 1-15 (1—being the low end, 15—being the high end).

More particularly, speed of oil out was assessed as follows: distributing the formulation in palms for 15 circles, if no oil was out within the 15 circles, a score of 0 was given; if oil was out within 1 circle, scored it 15.

Whiteness as emulsification was assessed as follows: adding 0.5 ml tap water on one palm, distributing water on the cheek with the formulation to be emulsified. If no white phenomenon appeared, a score of 0 was given; if the product turned to opal, then score it 15.

### Example 3: Results of the Evaluations of Example 2

3.1 Formulations 1 to 21 were observed for the appearance immediately after preparation and after storage at ambient temperature and elevated temperature. The results were shown in the following Table 5.

TABLE 5

| | | | | After storage | | | | | |
|---|---|---|---|---|---|---|---|---|---|
| | Immediately after | | | Method A | | | Method B | | |
| | preparation | | | (25° C., 3 months) | | | (50° C., 1 month) | | |
| | Trans.¹ | Col.² | Sep.³ | Trans.¹ | Col.² | Sep.³ | Trans.¹ | Col.² | Sep.³ |
| Formulation 1 | ○ | ○ | ○ | ○ | ○ | ○ | ○ | ○ | ○ |
| Formulation 2 | ○ | ○ | ○ | ○ | ○ | ○ | Δ | Δ | ○ |
| Formulation 3 | ○ | ○ | ○ | Δ | Δ | ○ | ▪ | ▪ | ▪ |
| Formulation 4 | ○ | ○ | ○ | ○ | ○ | ○ | ○ | ○ | ▪ |
| Formulation 5 | ○ | ○ | ○ | ○ | ○ | ▪ | Δ | Δ | ▪ |
| Formulation 6 | ○ | ○ | ○ | Δ | Δ | ○ | ▪ | ▪ | ▪ |
| Formulation 7 | Δ | ○ | ○ | Δ | ○ | ○ | Δ | ○ | ○ |
| Formulation 8 | ○ | ○ | ○ | ○ | ○ | ○ | ○ | ○ | ○ |
| Formulation 9 | ○ | ○ | ○ | ○ | ○ | ○ | ○ | ○ | ○ |
| Formulation | ○ | ○ | ○ | ○ | ○ | ○ | ○ | ○ | ○ |
| 10 | | | | | | | | | |
| Formulation | ○ | ○ | ○ | ○ | ○ | ○ | ○ | ○ | ○ |
| 11 | | | | | | | | | |
| Formulation | ○ | ○ | ○ | ○ | ○ | ○ | ○ | ○ | ○ |
| 12 | | | | | | | | | |
| Formulation | ○ | ○ | ○ | ○ | ○ | ○ | ○ | ○ | ○ |
| 13 | | | | | | | | | |
| Formulation | ○ | ○ | ○ | ○ | ○ | ○ | ○ | ○ | ○ |
| 14 | | | | | | | | | |
| Formulation | ○ | ○ | ○ | ○ | ○ | ○ | Δ | Δ | Δ |
| 15 | | | | | | | | | |
| Formulation | ○ | ○ | ○ | ○ | ○ | ○ | Δ | Δ | ○ |
| 16 | | | | | | | | | |
| Formulation | ○ | ○ | ○ | ○ | ○ | ○ | Δ | Δ | ○ |
| 17 | | | | | | | | | |
| Formulation | ○ | ○ | ○ | ○ | ○ | ○ | ○ | ○ | ○ |
| 18 | | | | | | | | | |
| Formulation | ○ | ○ | ○ | ○ | ○ | ○ | ○ | ○ | ○ |
| 19 | | | | | | | | | |
| Formulation | ○ | ○ | ○ | ○ | ○ | ○ | ○ | ○ | ○ |
| 20 | | | | | | | | | |
| Formulation | ▪ | ○ | ○ | ▪ | ○ | ○ | ▪ | ▪ | ▪ |
| 21 | | | | | | | | | |

Note:
1: transparence, 2: color changing; 3: separation.

The above results as shown in Table 5 demonstrated that the inventive Formulations 1, 4, and 7-20 comprising the combination of the first and second non-ionic surfactants exhibited superior stability when compared with comparative Formulations 2, 3, 5, 6, 21—comprising either of the first and second non-ionic surfactants. More particularly, Formulation 1 of the invention showed no slight or remarkable changes in the appearance including transparence, color changing and separation after storage at both 25° C. for 3 months and at 50° C. for 1 month. In contrast, comparative Formulation 2 showed slight color changing but remarkable viscosity dropped after storage at 50° C. for two weeks, and comparative Formulation 3 showed slight color changing after storage at 25° C. for 3 months but showed remarkable separation after storage at 50° C. for one week. Formulation 4 of the invention showed no slight or remarkable changes in the appearance including transparence, color changing and separation after storage at 25° C. for 3 months. Despite the fact that Formulation 4 of the invention showed slight separation after storage at 50° C. for two weeks, it still had superior stability as compared with comparative Formulations 5 and 6 (wherein, Formulation 6 changed to white from transparency when storing at 25° C. for 3 months and showed remarkable separation after storage at 50° C. for only one day; Formulation 5 showed remarkable separation after storage at 25° C. for 3 months and after storage at 50° C. for only one day). Formulation 21 showed white opaque appearance immediately after preparation, and remarkable separation was found at 50° C. after one week. Formulation 16 and 17 were found a little hazy after putting at 50° C. for one month, while Formulation 18-20 showed no slight or remarkable changes in the appearance including transparence, color changing and separation after storage at both 25° C. for 3 months and at 50° C. for 1 month.

3.2 Formulations 1 to 3 were measured at 25° C. for the viscosity of samples after storage at 50° C. The results were shown in the following Table 6.

TABLE 6

| | Time | For- | For- | For- |
|---|---|---|---|---|
| | point | mulation 1 | mulation 2 | mulation 3 |
| Viscosity | Initial | 69000 | 63200 | 304000 |
| | (overnight) | | | |
| | 1 week | 50000 | 49000 | 296000 |
| | 2 weeks | 41200 | 27000 | n.d. |
| | 3 weeks | 42000 | 19000 | n.d. |
| | 4 weeks | 40000 | 10000 | n.d. |

Note:
n.d. means “not determined”.

As can be seen from the above results, the viscosity of Formulation 1 of the invention (comprising the combination of the first and second non-ionic surfactant) decreased from 69000 mPas to 40000 mPas after storage at 50° C. for 4 weeks, which was still within the desirable range. After storage at 50° C. for 4 weeks, Formulation 1 of the invention showed no change in transparence and color, and especially no separation was observed, indicating that the decrease of the specific viscosity values was acceptable. In contrast, comparative Formulation 2 (comprising Lauryl glucoside alone as the surfactant portion) showed significant decrease of viscosity from 63200 mPas to 10000 mPas, and comparative Formulation 3 (comprising Oleth-20 alone as the surfactant portion) had a very high viscosity up to 304000 at initial but the viscosity sharply decreased after 1 week and showed significant separation to the extent that no viscosity value could be determined.

3.3 Formulations 1 to 6 were subject to the freeze-thaw test. The more cycles a formulation could stand, the more stable it was. The results were as follows: Formulation 1-3 passed three freeze-thaw cycles without separation. Formulation 6 (comprising the second non-ionic surfactant—Oleth-20—alone) separated after only one freeze-thaw cycle, whereas Formulation 5 (comprising the first non-ionic surfactant—Coco-glucoside—alone) and Formulation 4 (comprising the combination of the first and second non-ionic surfactants—Oleth-20 and Coco-glucoside) passed three freeze-thaw cycles without separation. Formulation 16-20 were also subject to the free-thaw test, all of which passed three freeze-thaw cycles without separation. Formulation 21, which used PEG-20 glyceryl stearate alone, failed the freeze-thaw test.

3.4 Formulations 1 to 3 and Formulations 12, 15 were subject to the make-up cleansing test. The make-up removal efficacy (%) before cleansing with each formulation was set 0% (as benchmark), and the removal efficacy (%) after cleansing with each formulation was shown as mean±SD in the following Table 7. The higher the removal efficacy was, the higher removal capability the formulation had. All the samples were tested in their steady state without separation. Each test was repeated three times to calculate average score.

TABLE 7

| Removal | The surfactant | | |
|---|---|---|---|
| efficacy (%) | portion | For lipstick | For foundation |
| Formulation 1 | 50% 1200UP + | 76.49 ± 0.01 | 82.89 ± 0.02 |
| | 50% O20S | | |
| Formulation 2 | 100% 1200UP | 58.99 ± 0.03 | 80.80 ± 0.03 |
| Formulation 3 | 100% O20S | 75.45 ± 0.03 | 77.73 ± 0.02 |
| Formulation 12 | 30% 1200UP + | 79.44 ± 0.03 | 88.15 ± 0.04 |
| | 70% O20S | | |
| Formulation 15 | 70% 1200UP + | 83.55 ± 0.01 | 90.61 ± 0.03 |
| | 30% O20S | | |

Note:
the column of the surfactant portion indicates the surfactants comprised in the tested formulations. The 1200UP refers to the PLANTACARE ® 1200 UP and O20S refers to EUMULGIN ® O 20 S. The percentage before 1200UP indicates the percentage of the active ingredient Lauryl glucoside based on the total weight of the surfactant portion, and the percentage before O20S indicates the percentage of the active ingredient Oleth-20 based on the total weight of the surfactant portion.

From the above results of make-up removal efficacy, it can be seen that Formulations 1, 12 and 15 of the invention comprising the combination of the first and second non-ionic surfactants exhibited superior removal capability when compared with comparative Formulations 2 and 3 comprising either of the first and second non-ionic surfactants.

3.5 Formulations 1 to 3 were subject to the in-use sensory test. The results were shown in the following Table 8.

TABLE 8

| | | Formulation | Formulation | Formulation |
|---|---|---|---|---|
| Test item | | 1 | 2 | 3 |
| Before | Transparency | 10.8 | 8.6 | 5.3 |
| use | Ease to Pick up | 9.9 | 9.7 | 5.5 |
| In use | Speed of oil out | 10.3 | 8.0 | 10.4 |
| After use | Whiteness as | 9.2 | 8.8 | 8.8 |
| | emulsification | 8.5 | 9.2 | 7.5 |
| | Ease of rinsing | | | |
| | Wet slippery | 9.5 | 9.9 | 9.6 |
| | Wet stickiness | 4.5 | 4.6 | 4.5 |
| | Skin tightness | 6.5 | 7.5 | 7.3 |
| | (instant) | 6.5 | 7.8 | 7.7 |
| | Skin dryness | | | |
| | (instant) | 8.8 | 8.6 | 8.1 |
| | Smoothness | | | |
| | (instant) | | | |
| | Skin tightness | 6.9 | 7.9 | 7.5 |
| | (2 min) | | | |
| | Skin dryness | 6.8 | 7.9 | 7.5 |
| | (2 min) | | | |
| | Smoothness | 9.2 | 8.9 | 8.6 |
| | (2 min) | | | |

As can be seen from the above scoring results, Formulation 1 of the invention showed the best transparence appearance having a score much higher than comparative Formulations 2 and 3, which was more attractive for consumers. As compared with comparative Formulations 2 and 3, Formulation 1 of the invention was easy to pick up, and exhibited the best emulsification helping to remove make-up during use, which was much convenient and pleasing for consumers. When compared with Formulation 2, Formulation 1 showed superior speed of oil out, and less wet slippery and less wet stickiness which were linked to better cleansing feel for consumer use. In addition, as compared with comparative Formulations 2 and 3, Formulation 1 showed the least skin tightness and dryness after use, and the best smoothness, which would provide lasting smoothness and moisturization to the skin and thus was pleasing for consumers. When compared with comparative Formulations 2 and 3 comprising either of the first and second non-ionic surfactants, Formulation 1 of the invention comprising the combination of the first and second non-ionic surfactants had the best transparence appearance before use, and provided superior pleasant skin sensory during use and after use. On the whole, for Formulation 1 of the invention, good performance was observed on all the tested items throughout the entire process of consumer use including the before-use, in-use and after-use stages. Formulation 18 comprising polyoxyethylene fatty acid glyceryl esters as the second nonionic surfactant showed similar sensory trends as Formulation 1. Formulation 18 has good transparency appearance and delivered satisfactory sensory during and after use.

In sum, the cosmetic compositions of the invention, comprising the combination of the first non-ionic surfactant and the second non-ionic surfactant, were in the form of transparent or semi-transparent gel, had high oil content, exhibited superior stability at both ambient temperature and elevated temperature, showed desirable cleansing efficacy, provided lasting smoothness and moisturization to the skin, and thus were pleasing and attractive to consumers.

1. A cosmetic composition, comprising
(a) an oil phase;
(b) a surfactant portion comprising a first non-ionic surfactant and a second non-ionic surfactant, wherein the first nonionic surfactant comprises at least one alkyl polyglucoside and the second nonionic surfactant is at least one selected from a surfactant having the following general formula (I) or (II):
R(X)ₙOR′ (I)
wherein, R represents a linear or branched C₈-C₃₀ alkyl group or C₈-C₃₀ alkenyl group;
X represents —OCH₂CH₂— or —OCH₂—CHCH₃—;
n represents an integer from 2 to 100; and
R′ represents H, or a linear or branched C₈-C₃₀ alkyl group or C₈-C₃₀ alkenyl group; 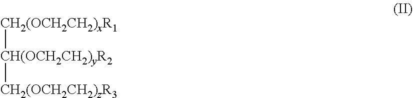
wherein, the sum of x+y+z represents an integer from 2 to 100;
and R₁, R₂, and R₃, are the same or different and each independently represents OH, and/or a linear or branched C₈-C₃₀ fatty acid ester group, with the proviso that at least one of R₁, R₂ and R₃ is not OH;
(c) an aqueous phase; and
(d) at least one C₂-C₆ polyol;
wherein said composition is in the form of a transparent or semi-transparent gel.

2. The cosmetic composition of claim 1, wherein based on the total weight of the composition,
(1) the oil phase is present in an amount of from 50 wt % to 90 wt %;
(2) the surfactant portion comprising the first non-ionic surfactant and the second non-ionic surfactant is present in an amount of from 1 wt % to 20 wt %;
(3) the aqueous phase is present in an amount of from 1 wt % to 15 wt %; and
(4) the C₂-C₆ polyol is present in an amount of from 1 wt % to 15 wt %.

3. The cosmetic composition of claim 1, wherein the first non-ionic surfactant is present in an amount of from 0.5 wt % to 19.5 wt %; and the second non-ionic surfactant is present in an amount of from 0.5 wt % to 19.5 wt %; based on the total weight of the composition.

4. The cosmetic composition of claim 1, wherein the alkyl polyglucoside is one or more selected from the group consisting of C₈-C₁₀ alkyl polyglucosides, C₈-C₁₆ alkyl polyglucosides, C₁₂-C₁₆ alkyl polyglucosides, C₉-C₁₁ alkyl polyglucosides, C₁₂-C₂₀ alkyl polyglucosides, and a combination thereof.

5. The cosmetic composition of claim 1, wherein in the general formula (I):
R represents a linear or branched C₁₀-C₃₀ alkyl group or C₁₀-C₃₀ alkenyl group;
X represents —OCH₂CH₂—;
n represents an integer from 3 to 90; and
R′ represents H, or a linear or branched C₁₀-C₃₀ alkyl group or C₁₀-C₃₀ alkenyl group.

6. The cosmetic composition of claim 1, wherein in the general formula (I):
R represents a linear or branched C₁₂-C₂₄ alkyl group or C₁₂-C₂₄ alkenyl group;
X represents —OCH₂CH₂—;
n represents an integer from 2 to 30; and
R′ represents H.

7. The cosmetic composition of any of claim 1, wherein in the general formula (II): 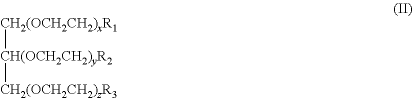
the sum of x+y+z represents an integer from 3 to 90; and
R₁, R₂, and R₃, are the same or different and are each independently represents OH, and/or a linear or branched C₁₀-C₃₀ fatty acid ester group, with the proviso that at least one of R₁, R₂ and R₃ is not OH.

8. The cosmetic composition of claim 1, wherein in the general formula (II): 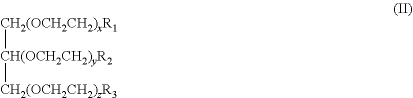
the sum of x+y+z represents an integer from 2 to 30; and
R₁, R₂, and R₃, are each the same or different and are each independently represents OH and/or a linear or branched C₁₂-C₂₄ fatty acid ester group, with the proviso that at least one of R₁, R₂ and R₃ is not OH.

9. The cosmetic composition of claim 1, wherein the second nonionic surfactant comprises at least one selected from the group consisting of polyoxyethylene ethers of lauryl alcohol, macrogol lauryl ethers, polyoxyethylene ethers of cetyl alcohol, polyoxyethylene ethers of cetylstearyl alcohol, polyoxyethylene ethers of stearyl alcohol, polyoxyethylene ethers of oleyl alcohol, polyoxyethylene tridecyl ethers, and a combination thereof.

10. The cosmetic composition of claim 1, wherein the second nonionic surfactant comprises at least one of the following:
polyoxyethylene (4) lauryl ether (Laureth-4, INCI);
polyoxyethylene(9) lauryl ether (Laureth-9, INCI);
polyoxyethylene (23) lauryl ether (Laureth-23, INCI);
polyoxyethylene (2) cetyl ether (Ceteth-2, INCI);
polyoxyethylene (10) cetyl ether (Ceteth-10, INCI);
polyoxyethylene (15) cetyl ether (Ceteth-15, INCI);
polyoxyethylene (20) cetyl ether (Ceteth-20, INCI);
polyoxyethylene (30) cetyl ether (Ceteth-30, INCI);
polyoxyethylene (6) cetylstearyl ether (Ceteareth-6, INCI);
polyoxyethylene (12) cetylstearyl ether (Ceteareth-12, INCI);
polyoxyethylene (20) cetylstearyl ether (Ceteareth-20, INCI);
polyoxyethylene (25) cetylstearyl ether (Ceteareth-25, INCI);
polyoxyethylene (30) cetylstearyl ether (Ceteareth-30, INCI);
polyoxyethylene (2) stearyl ether (Steareth-2, INCI);
polyoxyethylene (10) stearyl ether (Steareth-10, INCI);
polyoxyethylene (20) stearyl ether (Steareth-20, INCI);
polyoxyethylene (21) stearyl ether (Steareth-21, INCI);
polyoxyethylene (30) stearyl ether (Steareth-30, INCI);
polyoxyethylene (2) oleyl ether (Oleth-2, INCI);
polyoxyethylene (10) oleyl ether (Oleth-10, INCI);
polyoxyethylene (15) oleyl ether (Oleth-15, INCI);
polyoxyethylene (20) oleyl ether (Oleth-20, INCI);
polyoxyethylene (23) oleyl ether (Oleth-23, INCI);
polyoxyethylene (30) oleyl ether (Oleth-30, INCI);
polyoxyethylene (25) octyldodecyl ether;
polyoxyethylene (25) decyltetradecyl ether;
polyoxyethylene(9) C₁₂₋₁₄ secondary alkyl ether;
polyoxyethylene(12) C₁₂₋₁₄ secondary alkyl ether;
polyoxyethylene (20) behenyl ether (Beheneth-20, INCI);
polyoxyethylene (25) behenyl ether (Beheneth-25, INCI);
polyoxyethylene (30) behenyl ether (Beheneth-30, INCI);
polyoxyethylene (10) tridecyl ether (Trideceth-10, INCI);
polyoxyethylene (12) ether of glyceryl laurate;
polyoxyethylene (20) ether of glyceryl laurate;
polyoxyethylene (30) ether of glyceryl laurate;
polyoxyethylene (20) ether of glyceryl cocoate;
polyoxyethylene (30) ether of glyceryl cocoate;
polyoxyethylene (10) ether of glyceryl isostearate;
polyoxyethylene (20) ether of glyceryl isostearate;
polyoxyethylene (30) ether of glyceryl isostearate;
polyoxyethylene (10) ether of glyceryl diisostearate;
polyoxyethylene (20) ether of glyceryl diisostearate;
polyoxyethylene (30) ether of glyceryl diisostearate;
polyoxyethylene (10) ether of glyceryl oleate;
polyoxyethylene (20) ether of glyceryl oleate;
polyoxyethylene (30) ether of glyceryl oleate;
polyoxyethylene (15) ether of glyceryl ricinoleate;
polyoxyethylene (20) ether of glyceryl ricinoleate;
polyoxyethylene (10) ether of glyceryl stearate;
polyoxyethylene (20) ether of glyceryl stearate;
polyoxyethylene (30) ether of glyceryl stearate;
polyoxyethylene (10) ether of glyceryl triisostearate;
polyoxyethylene (20) ether of glyceryl triisostearate;
polyoxyethylene (30) ether of glyceryl triisostearate;
polyoxyethylene (10) ether of glyceryl trioleate;
polyoxyethylene (20) ether of glyceryl trioleate;
polyoxyethylene (30) ether of glyceryl trioleate;
polyoxyethylene (10) ether of glyceryl tristearate; and
polyoxyethylene (20) ether of glyceryl tristearate.

11. The cosmetic composition of claim 1, wherein the aqueous phase comprises water, and optionally one ore more water miscible solvents.

12. The cosmetic composition of claim 1, wherein the C₂-C₆ polyol is one ore more selected from the group consisting of glycerin, propanediol, butanediol, and sorbitol.

13. The cosmetic composition of claim 1, wherein the oil phase comprises one or more cosmetically acceptable oil or mixtures thereof.

14. The cosmetic composition of claim 1, wherein the composition is free or substantially free of polymeric thickener or gelling agent or thickening polymer.

15. The cosmetic composition of claim 14, wherein an amount of the polymeric thickener or gelling agent or thickening polymer, if present, is present in an amount of lower than 5 wt % of the cosmetic composition.

16. The cosmetic composition of claim 1, wherein the composition optionally further comprises one or more surfactants selected from the group consisting of anionic surfactants, amphoteric surfactants and cationic surfactants, and mixtures thereof, provided they are compatible with the above first and second non-ionic surfactants.

17. The cosmetic composition of claim 1, wherein the composition optionally further comprises additives generally blended in a cosmetic composition.

18. A method for preparing the cosmetic composition of claim 1, comprising:
(a) adding the surfactant portion, the C₂-C₆ polyol to the aqueous phase, and mixing, optionally with heating, stirring, or simultaneous heating and stirring until uniform;
(b) heating the oil phase;
(c) adding the oil phase obtained in step (b) into the aqueous phase obtained in step (a) under stirring; and
(d) cooling the mixture obtained in step (c) under stirring to give a gel.

19. The method of claim 18, wherein the aqueous mixture in step (a) is heated to a temperature in the range of 50° C.-80° C., and the oil phase in step (b) is heated to a temperature in the range of 50° C.-80° C.

20. The method of claim 18, wherein in step (c), the adding rate of the oil phase into the aqueous phase is in the range of from 1 wt % to 8 wt % of the oil phase per minute.

21. The cosmetic composition of claim 1 used for the preparation of personal care products.

22. A method for personal care or cleansing, comprising applying the cosmetic composition of claim 1 to a desired location on the skin or hair in the presence of water, and then rinsing the composition from the skin or hair with water.

23. The method of claim 22, wherein after being applied to the skin, the composition produces cleansing foams.

24. The method of claim 22, wherein the method is a facial cleansing method and the composition is applied to at least one part of the face.

25. The cosmetic composition of claim 4 wherein the alkyl polyglucoside is one or more selected from the group consisting of decyl glucoside, arachidyl glucoside, caprylyl/capryl glucoside, cetearyl glucoside, coco-glucoside, lauryl glucoside, and a combination thereof
